(19) United States
(12) Patent Application Publication
(10) Pub. No.: US 2010/0197544 A1
(43) Pub. Date: Aug. 5, 2010

(54) Rinseless body wash composition

(76) Inventor: Edwin De La Cruz, New York, NY (US)

Correspondence Address: Ashley J. Wells, 27 Outlook Circle, Swannanoa, NC, 28778, US

(21) Appl. No.: 12/322,428

(22) Filed: Feb. 3, 2009

(51) Int. Cl. A61K 8/97; A61Q 19/10

(52) U.S. Cl. 510/157

(57) ABSTRACT

A body wash composition that does not require rinsing and that topically cleanses a biological entity includes from about 0.5% to about 20.0% by weight of a concentrated extract of Arnica; and remainder pure water. The composition may further include a foaming agent, and optionally, humectants, and/or fragrance. Preferably, the concentrated extract of Arnica may be one of an infusion of Arnica flowers in pure water or a tincture of Arnica flowers in alcohol, and is present in an amount effective to provide antimicrobial properties sufficient to reduce body odor of a biological entity. A method of externally cleansing a body of a biological entity includes applying the body wash composition externally to the body. The composition is suitable for cleansing sensitive areas of the body including genital and anal areas even when allowed to dry in place.

### CROSS-REFERENCE TO RELATED APPLICATIONS

This non-provisional Application claims the benefit of the priority of Applicant's earlier filed U.S. Provisional Application Ser. No. 61/025,336 filed on Feb. 1, 2008, the entire contents of which are incorporated herein by reference.

### BACKGROUND OF THE INVENTION

1.Field of the Invention

The invention relates to a body wash composition and, more particularly, to a body wash composition that is applied topically to cleanse and mask and/or deodorize a biological entity, human and animal, but does not require rinsing with water and may be left in place with no harmful effects.

2. Description of the Related Art

Campers, military personnel, astronauts, and people experiencing natural or man-made disasters, such as war and internment, quickly become aware of our need for water for personal hygiene. Even one day without the modern convenience of tap water on demand is stressful such as when a water main breaks. Today, the availability of clean water is frequently in the news in developed and developing areas of the world, and has become a world-wide concern due to changing weather patterns, pollution, and even the specter of terrorist activities.

Indeed, personal hygiene is a daily basic human necessity. The practice has been accepted socially in ancient cultures such as those in China and Japan for thousands of years. In Western civilizations, ancient Rome was pro-hygiene and bathing was practiced and considered socially necessary

In hospital environments, the traditional method of personal hygiene for bed-ridden patients has been “sponge baths” in which soap and water is applied by a caregiver followed by rinsing with pure water, drying, and application of emollients. For localized debris, a wet cloth or towel has been long employed followed by drying and application of emollients. Alcohol rubs generally using isopropanol have been enjoyed by patients for their refreshing benefits.

In the second half of the 20^{th} century, waterless compositions that do not requiring rinsing with water began to be employed as an alternative to the basic and traditional soap and water method. This rinseless technology reduced the amount of time and minimized movement of the patient needed to bathe the patient, and reduced the risk of water-borne disease that was determined to exist and require management in public health institutions, such as hospitals and elder care facilities. As hospital populations increased so did the need for a waterless composition. At first, alcohol-based liquid compositions were use, and these were followed by the convenience of pre-moistened towelettes and wipes.

Public health institutions, such as hospitals and elder care facilities, have solved the need to bathe patients confined to bed using body wash formulations applied either directly as a liquid or diluted with water prior to application, as well as using pre-moistened wipes for cleansing smaller body areas. Campers, military personnel, astronauts, and parents with small children have resorted to similar solutions. Such body wash formulations and pre-moistened wipes generally contain synthetic ingredients including antimicrobials and many are based on alcohol. While effective for topical use, such formulations can be toxic to varying degrees due to the nature of the constituents.

U.S. Pat. No. 5,098,694 to Komp, the disclosure of which is incorporated herein by reference, describes two body odor elimination compositions including deodorants and antimicrobial ingredients for external use on the human body. In one composition, from 0.1-0.5% by weight of nonionic deodorant ingredients include glyceryl laurate, sorbic acid, and citric acid. An important function of such compositions is to mask body odor caused by bacterial action upon perspiration.

Researchers have found, however, that many of the ingredients typically used in prior art cosmetic formulas, e.g., cleansing compositions, can be toxic and have adverse immediate and/or cumulative effects on the health of biological entities, such as allergic reactions and immune system impairment.

For example, Cosmetic Ingredient Review has published an assessment that indicates that benzalkonium chloride is a human skin toxicant. Benzalkonium chloride has been used in cosmetic formulations as an antimicrobial agent, a deodorant, a preservative, an anti-static agent, and a surfactant.

As a preservative, many, if not most, rinseless formulations employ ingredients from the paraben family due to their significant bactericidal and fungicidal properties. Most members of the paraben family have been found to have negative health consequences. For example, propyl paraben is considered toxic to the human developmental and reproductive system. Moderate concerns for this ingredient include allergies, immunotoxicity, endocrine disruption, and organ system toxicity. A published Cosmetic Industry Review concluded that propyl paraben sensitizes the skin and can instigate an immune system response including itching, burning, scaling, hives, and blistering. In comparison, methyl paraben is also toxic but with slightly different consequences. Methyl paraben can contribute to cancer. Further, toxicology research has concluded that low dose consumption in animals showed broad systemic effects at low doses. In the Journal of Applied Toxicology, (1): 67-77, Jan.-Feb. 27, 2007, article titled “Comparison of the global gene expression profiles produced by methylparaben, n-butylparaben and 17 beta-oestradiol in MCF7 human breast cancer cells”, D. Pugazhendhi, A. J. Sadler, and P. D. Darbre conclude that the parabens tested had negative biochemical or cellular level changes including interference with gene expression.

Diazolidinyl urea obtained from animal urine has been used as a preservative in cosmetic formulations. Multiple research studies of diazolidinyl urea have reported positive mutation of cancer in one or more in vitro tests on mammalian cells. Other health related consequences reported include allergies and immunotoxicity.

As an additional example, benzyl alcohol has been used as a odor masking agent in antimicrobial cleansers. Multiple sources report that benzyl alcohol has moderate health consequences. In the Journal of Applied Toxicology, (1): 67-77, Jan.-Feb. 27, 2007, article titled “Comparison of the global gene expression profiles produced by methylparaben, n-butylparaben and 17 beta-oestradiol in MCF7 human breast cancer cells”, D. Pugazhendhi, A. J. Sadler, and P. D. Darbre, benzyl alcohol can cause neurotoxicity of the human skin. Other findings conclude that it can contribute to cancer, development/reproductive toxicity, allergies and immunotoxicity, neurotoxicity, organ system toxicity, and irritation of the skin, eyes, and lungs

Propylene glycol finds multiple uses in cosmetic formulas as an humectant, a skin conditioner, a fragrance, and a viscosity adjusting agent. Propylene glycol has been reported to cause multiple health problems, however. Research reported in the Journal of Pharmaceutical Therapy, titled “Propylene Glcol: The safe diluent that continues to cause harm”, Vol. 16, pgs. 690-703, Dec. 19, 1996, concluded that propylene glycol can negatively affect the human general nervous system. Other findings report a relation to the incidence of cancer, developmental and reproductive toxicity, allergies and immunotoxicity, irritation of multiple organs (skin, eyes, or lungs), enhanced skin absorption, endocrine disruption, and neurotoxicity.

As final examples, solvents such as methyl alcohol, ethyl alcohol, and isopropyl alcohol have been found to dry the skin, particularly with prolonged use. Additionally, the benefits of soap-free and petrolatum-free compositions in relation to skin cleansing can be found in U.S. Pat. No. 5,801,134 to Righton and U.K. Patent No. 9,413, 931 to Righton, which are incorporated herein by reference.

In view of the foregoing needs, it is an object of this invention to provide a body wash composition that feels wet and cleanses biological entities, and effectively masks and/or eliminates odor.

It is yet another object of this invention to provide a body wash composition that does not require rinsing with water, that is, is rinseless, and is safe to leave on the body even after prolonged use.

It is still another object of this invention to provide a body wash composition that is a simple mixture of readily available ingredients and cost-effective to produce because it contains a minimum number of modestly priced ingredients.

It is a further object of this invention to provide a body wash composition which lathers, i.e., foams, when rubbed between the hands so that the foam feels wet to the user and a small amount of the composition covers a large area when foamed.

### SUMMARY OF THE INVENTION

These and other objects are accomplished by the present invention which provides a body wash composition that does not require rinsing and that topically cleanses a biological entity, comprising: from about 0.5% to about 20.0%, preferably from about 2.0% to about 15.0%, more preferably from about 2.0% to about 5.0%, by weight of a concentrated extract of Arnica; and remainder pure water. The concentrated extract of Arnica may be one of an infusion of Arnica flowers in pure water or a tincture of Arnica flowers in alcohol, and is present in an amount effective to provide antimicrobial properties sufficient to reduce body odor of a biological entity.

The body wash composition may further comprise from about 0.05% to about 5% by weight of a foaming agent. The foaming agent is preferably a nonionic surfactant. The foaming agent may be at least one of sodium laureth sulfate, cocamide DEA, cocamidopropyl betaine, cocamide betaine, coco betaine, natural coconut oil, lauramide MEA, decyl glucoside, cocamide methyl MEA, and Polysorbate 80 acetate. Preferably, the foaming agent is CALBLEND® CLEAR manufactured by The Pilot Chemical Company, U.S.A. The foaming agent produces a foam when rubbed onto hands and other body parts of the biological entity so that the foam feels wet to the biological entity and a small amount of the body wash composition covers a large area.

The body wash composition may further comprise from about 1.0% to about 15.0% by weight of a humectant. The humectant may include glycerine or aloe vera gel.

The body wash composition may further comprise from about 0.25% to about 2.0% by weight of fragrance.

The body wash composition may further comprise from about 0.5% to about 10.0% by weight of additional ingredients including at least one of Vitamin E, aloe vera gel, tea tree extract, peppermint extract, anis extract, valerian extract, lemongrass oil, calendula extract, nettle extract, lavender extract, rose extract, lemon juice, lime juice, and grapefruit juice.

The body wash composition has a pH value of ranging from 5.5 (the pH of normal skin) to 9.0. The body wash composition preferably has an appearance that is clear and colorless, a smell that is pleasant, evaporates in 30 seconds or less, and does not clog pores of the biological entity's skin.

The objects of the invention are accomplished by providing a body wash composition that does not require rinsing and that topically cleanses a biological entity, comprising, in percent by weight: 2.0% of a concentrated extract of Arnica; 1.0% of a foaming agent which is CALBLEND® CLEAR; 1.0% of cocamide from natural coconut; 1.0% of glycerin from a vegetarian source; 0.1% of a fragrance; and remainder pure water.

These and other objects are accomplished by the present invention which provides a method of externally cleansing a body of a biological entity, comprising: (a) providing a body wash composition that does not require rinsing and that topically cleanses a biological entity, comprising: from about 0.5% to about 20.0%, preferably from about 2.0% to about 15.0%, more preferably from about 2.0% to about 5.0%, by weight of a concentrated extract of Arnica; and remainder pure water; and (b) applying the body wash composition externally to the body of the biological entity. The step of applying may include applying to sensitive areas of the body including genital and anal areas even when allowed to dry in place. The method may further comprise at least one of removing by toweling with a cloth and allowing the body wash composition to air dry.

### DETAILED DESCRIPTION OF THE INVENTION

The body wash cleansing product of this invention is for personal hygiene use, contains no soap, no animal products or by-products so as to be suitable for use by vegetarians, and is not tested on animals or human subjects since no FDA approval is required. It is water-based and feels wet to biological entities and is safe to use. Applied topically as a body scrub or wash, it has a cleansing effect which effectively masks and/or eliminates odor. It does not require rinsing with water; that is, it is rinseless, and is non-irritating to most users, even when allowed to dry in place. It is a simple mixture of readily available ingredients and cost-effective to produce because it contains a minimum number of modestly priced ingredients.

Preferably, the body wash composition lathers, i.e., foams, when rubbed on the body or between the hands and then applied to the body, so that the foam not only feels wet to the user but a small amount of the composition covers a large area when foamed. The foam has a beneficial cleansing effect on the body since it acts to speed removal of external dust, dirt, and cosmetics, as well as biological waste including sweat, body oils, urine and excrement. The foam leaves the skin feeling fresh with no soap or detergent residues. Scrubbing the body with foams have positive health related effects as discussed in U.S. Pat. No. 6,800,597 to Campagnoli, the disclosure of which is incorporated herein by reference, because foams remove dead skin cells from the body allowing better nutrition and oxygenation of the skin.

The composition is water-based and includes an herbal antimicrobial diluted with pure water. In preferred embodiments, the composition is a simple mixture additionally including a foaming agent, and optionally humectants to maintain consistency, and a very light perfume to provide consumer appeal. The proportions are not critical and will be discussed in the following.

It was determined that the botanical Arnica montana has natural anti-microbial and odor masking properties that are useful in the novel body wash composition of the present invention, particularly when the composition is used without rinsing. A mild herbal antimicrobial for a rinseless body wash composition is considered preferable to the previously-used antibacterial deodorants, such as phenolic ethers, quaternary ammonium compounds, hexachlorophene, ion exchange materials, and the like, which would be expected to be irritating to the body unless rinsed away with water. Furthermore, Arnica montana is a mild antimicrobial and does not appear to negatively influence the natural microbial flora of the skin so that the biological equilibrium of the skin is not interrupted. Accordingly, the herbal antimicrobial selected for the body wash composition of the present invention is Arnica montana, which is also known a leopard's bane, wolf bane, mountain tobacco, mountain arnica, and simply Arnica. As referenced herein, the common name, “Arnica”, is used to signify Arnica montana. Additional herbal antimicrobials, such as oil of oregano, may be employed in addition to Arnica, but the amount of Arnica extract should be the major constituent, i.e., 50% by weight or more, for this mild antimicrobial function.

The botanical Arnica is a member of the N.O. Compositae (Asteraceace) family, and has daisy-like flowers. The plant is a perennial herb indigenous to Central Europe, North America and other colder mountain environments, but may be successfully cultivated elsewhere, and the roots and flowers have been used medicinally for centuries.

A gel made from the roots of the plant contains thymol and derivatives of thymol, and has been used as a topical medicinal. The multiple health related benefits include the use as a gel, which is considered to have anti-inflammatory benefits similar to NSAIDS (ibuprofen) in treating the symptoms of, for example, hand osteoarthritis. The gel additionally is a vasodilator of subcutaneous blood capillaries which facilitates transport of blood and fluids and speed healing of strains, sprains, and bruises. The gel has been used as a fungicide and preservative. Internal ingestion is contraindicated because the plant contains the toxin helenalin which can be poisonous if ingested in large amounts and dilution with water is necessary to prevent skin irritation from direct contact with the plant in some cases.

Arnica flowers have been reported to contain active constituents, such as sesquiterpene lactones, and extracts are known to provide a three-fold benefit as an anti-irritant, an anti-inflammatory, and an anti-bacterial as reported, for example, in Andrew Pengelly's text The Constituents of Medicinal Plants: An introduction to the chemistry and therapeutics of herbal medicine, 2^{nd} Ed., CABL Publishing, 2004, pgs. 62-63. Pengelly reports that a tincture of Arnica flowers contains sesquiterpene lactones including two main ingredients, helenalin and dihydorhelanin, which act as an anti-inflammatory when applied to the skin, thus providing an important medicinal benefit. Dihydrohelanin's ester derivatives have been reported to be able to inhibit cytokines TNF and IL-1 by regulation of their transcription factors, in a similar way to glucocorticoids. Varro E. Tyler, et al. in Tyler's Honest Herbal: sensible guide to the use of herbs and related remedies, 4^{th} Ed., The Haworth Herbal Press, NY, 1999, pgs. 39-40 and 402403, further reports that preparations made form Arnica flowers are believed to have antibiotic qualities and are safe and effective to use when applied topically. Topically applied Arnica extracts are known also to reduce the swelling and pain caused by insect bites, such as mosquito bites.

Extracts of Arnica roots and flowers can be infusions in water, tinctures in solvents, such as alcohol, and ointments in oil, such as, olive oil. Infusions and tinctures are useful in the body wash compositions of this invention. Reference herein to “an extract of Arnica” is meant to include infusions in water and/or tinctures prepared in any biocompatible solvent such as water, alcohol, and glycerin, and mixtures thereof, for example, a 95-5 water-alcohol mixture by volume or a 50-50 water-glycerin mixture by volume. For example, a water infusion may contain from 3 to 6% by weight of water-soluble constituents from either the roots or the flowers of Arnica. It is to be understood that the exact nature and content of constituents contained in Arnica roots and flowers varies from plant to plant so that an exact concentration for the extract of Arnica is difficult to obtain.

Reference herein to “a concentrated extract of Arnica” is meant to include an extract of Arnica roots and/or flowers, fresh or dehydrated, prepared in any solvent by any of the various extraction methods commonly employed so as to be substantially as concentrated as practical. As mentioned above, any biocompatible solvent such as water, alcohol, and glycerin, and mixtures thereof may be used by way of example but not limitation.

On a small scale, an infusion of Arnica flowers in water may contain from 3 to 6% by weight of water-soluble constituents and can be made as follows. Two to three teaspoons of chopped, fresh Arnica blossoms are placed in a warmed glass container. Two cups of fresh, non-chlorinated water are brought to a boil and added to the herbs. The container is covered and simmered for about 10 minutes. The liquid infusion is recovered by straining off the solids. The prepared infusion (tea) will store for about two days under refrigeration.

On an industrial scale, and by way of example but not limitation, batches of Arnica roots or blossoms harvested from the Arnica plant are comminuted, measured, and placed in a vessel. The roots and/or blossoms are preferably fresh, but dried roots and/or flower blossoms may be used while less preferred. Pure water is added to the vessel, either at room temperature or at a temperature ranging up to boiling, and the mixture is heated to maintain a temperature of at least 95° C., preferably of 96-100° C., more preferably 96-99° C., for up to about 1 hour, preferably 10 to 30 minutes, more preferably 10 to 15 minutes, to obtain a concentrated extract containing from 3 to 6% by weight of water-soluble constituents. The vessel may be a sealed container and heating may take place under an inert atmosphere, such as nitrogen or argon, or under a vacuum and at a lower temperature, to preserve the natural ingredients contained in the Arnica flowers.

As an herbal antimicrobial and /or odor masking ingredient in the novel body wash composition of this invention, from about 0.5% to about 20%, preferably from about 2.0% to about 15.0%, and most %, preferably from about 2.0% to about 5.0%, by weight of the concentrated extract of Arnica is prepared as described above and is further diluted with pure water up to 100% by weight. The herbal antimicrobial constituents contained in such a concentrated extract of Arnica successfully mask and/or eliminate body odors. This composition may be used as is, or additional constituents may be added and mixed in, such as foaming agents, humectants, fragrance, Vitamin E, and the like to enhance the composition's features.

Arnica has an astringent benefit and removes oil from the skin. In contrast to alcohols conventionally used for antimicrobial disinfecting purposes that prevent perspiration by closing the pores, the astringent, i.e., skin tightening, benefit of Arnica is obtained without perspiration being prevented. Additionally, including Vitamin E in the composition has been found to enhance the antimicrobial effect of the body wash composition without disturbing the skin's biological equilibrium.

The body wash composition advantageously includes from about 0.05% to about 5.0% by weight of a foaming agent which may be a nonionic surfactant. Most nonionic surfactants are considered safe and include, by way of example but not limitation, sodium laureth sulfate, cocamide DEA, cocamidopropyl betaine, cocamide betaine, coco betaine, natural coconut oil, lauramide MEA, decyl glucoside, cocamide methyl MEA, Polysorbate 80 acetate, and a proprietary blend of sodium laureth sulfate, cocamide DEA, and cocamidopropyl betaine commercially available as CALBLEND® CLEAR manufactured by The Pilot Chemical Company, U.S.A.

Foaming agents have a foaming action that is triggered by, for example, rubbing the composition, provided as a liquid or impregnated into a pre-moist wipe or towelette, between the hands or directly on the skin to produce the foam. Alternately, the foam may be produced by simply shaking a container of the liquid. Foam enhances the delivery and cleaning action of the composition. Delivery is enhanced by including a foaming agent in the composition because a foam includes air so that less product by weight is needed to cover the areas being cleansed while continuing to produce the desired wet feeling associated with cleansing. In a foam state, the body wash composition removes body impurities by the friction of rubbing, by simple dilution, and even by emulsification.

Ingredients to be added to the Arnica and water are selected not only for safe use, particularly when allowed to dry in place by evaporation instead of being toweled or rinsed off, but also with a view to availability at a low cost formulation by simple mixing together of ingredients. Ingredients can be mixed into the composition as solids, preferably as powders or other finely divided forms, or liquids, and solubility in water is preferred so that settling does not occur over time and so that emulsifiers are not required to maintain a suspension. It is desirable to minimize the number of ingredients. Embodiments containing emulsifiers, such as, by way of example but not limitation, pink grapefruit juice, may be used. That is, pink grapefruit juice, preferably freshly prepared, is a natural emulsifier for oily ingredients and may be included in amounts effective to prevent settling of the body wash composition.

Ingredients for the body wash composition are selected for a “good clean” based on synergy effects which may be thought of as including three stages. (1) The composition contacts the skin and removes impurities by rubbing, dilution, and emulsification. (2) Similarly, dead skin cells containing, for example, the keratin protein, are removed from the epidermis to assist in removal of pollutants clogging the pores. (3) The astringent effect of the Arnica then tightens the skin but without preventing perspiration. These enhance nutrition and oxygenation of the skin with obvious health benefits.

The body wash composition does not include petrolatum, beeswax, mineral oils, and/or other ingredients that would clog the skin's pores and prevent or impair perspiration and transpiration. Ingredients for the body wash composition may be selected to satisfy Islamic religious requirements prohibiting alcohol consumption in any manner and animal rights concerns regarding slaughter and testing.

### Methods of Use

Two methods or systems for use are contemplated.

(1) The most cost-effective packaging for the liquid body wash composition is a container, such as a bottle. Individual packettes enclosing measured portions of liquid may be used which, while more costly to fabricate, lend portability and the counterbalancing economy of measured portions. If refrigeration is not available, the bottle size should range from about 8 to 12 ounces so that it is used up before oxidation or rancidity become a problem, such as in one use. The bottle should be sealed until used. To dispense the liquid from a bottle, a pump, squeezable valve, or a removable screw cap may be used. The liquid composition is dispensed onto either one or both of (a) a palm of one hand and (b) a cloth such as a wash cloth as a means to transport the liquid to a desired area of the body, in a minimal amount, for example, an amount of up to about one ounce. When contact is made between liquid and the body, robust rubbing is suggested in a vertical, horizontal or circular motion, to effectively clean the body. When the composition includes a foaming agent, the robust rubbing should be effective to develop foam which is then distributed by further rubbing to effectively clean the body including the hair. The user or health care assistant then dries the body with a clean cloth, towel or paper napkin, and/or allows the residual composition to evaporate.

(2) The body wash composition may be impregnated into carrier means such as cloth or fabric sections for wipes. A set of wipes is then packaged into a sealed pouch holding, for example, about 8 wipes, which is generally sufficient to cleanse all of the areas of the body, except the hair. The end-user or assistant removes a fresh pre-moist wipe from the package for use on each major area of the body, such as the face, the left arm, the right arm, the left leg, the right leg, the genital area, the chest and stomach sections, and the anal area.

When contact is made between the composition and body, both methods of use work in similar ways on the body proper and achieve the same clean result of leaving the skin with a wet feeling. The container of liquid method is generally needed to cleanse the hair of the head for obvious reasons. The wetness of the composition and/or foam can be removed by toweling or allowing the composition to evaporate.

The method of use may be schematically illustrated by the following.

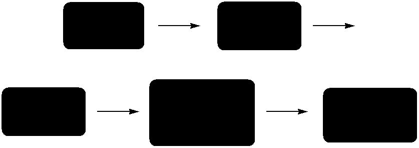

### Product Features and Structure

The body wash composition serves to replace water and preferably is a transparent liquid that appears to be clear, colorless, and transparent like water to simulate the physical appearance of water. When packaged in a bottle or another transparent container, preferably the amount of foaming agent used in an embodiment including a foaming agent is small enough that there is little or no foam reserve visible within the contents of the bottle. In the article titled “Eager Sellers and Stony Buyers: Understanding the Psychology of New Product Adoption” by John T. Gourville, Harvard Business Review, pages 98-106 June 2006, the author advised that humans are creatures of habit so that it is advisable that new products resemble and/or have the same qualities as the old product being replaced in the market-place. Thus, this appearance is expected to be more acceptable to the user.

Humectants, such as natural glycerin, may be included in the body wash composition to maintain the constitution, e.g., the moisture content, of the composition and to promote a fresh feeling for the skin after the composition is allowed to evaporate from the skin or is wipe off from the skin. Humectants from botanical sources are preferred, such as natural glycerin or Aloe Vera derived from vegetable sources, as is appreciated by vegetarians. From about 1.0% to about 15.0% by weight of humectants may be included in the composition.

Fragrances may be included in the composition and are preferably derived from botanicals extracted in water or alcohol. From about a trace, e.g., about 0.04%, to about 5.0%, preferably from about 0.1% $ to 2.0%, more preferably from about 0.1 to 0.25%, by weight of the natural water extract may be included. A very light scent that conveys freshness is preferred, such as citrus, for example, lemon or lime, or a scent that mimics the scent of soap or baby powder, or botanical-based essential oils, such as lavender, rose petals, calendula, peppermint, mint, nettle, spearmint, menthol, or any botanical essential oils that can be produced from any part of a plant including the blossom, seeds, woods, and leaves. The gentle fragrance preferably disperses in a brief period, for example, about 1 minute or less, preferably in about 10 to about 25 seconds.

The product preferably includes ingredients that are readily available, and, except for the Arnica, can be replaced with safe alternatives of natural or synthetic origin if war or natural disasters impairs supply lines, or if ingredient scarcity conditions occur. Manufacturing is preferably limited to simply mixing and packaging the ingredients and preferably less than six low cost ingredients including pure water are used

### EXAMPLE

A bio-based, rinseless body wash composition was prepared. The composition contained, in percent by weight:

2.0% of a concentrated Arnica extract. i.e., 3 to 5% by weight in water;

1.0% of CALBLEND® CLEAR, a proprietary blend of sodium laureth sulfate, cocamide DEA, and cocamidopropyl betaine manufactured by The Pilot Chemical Company;

1.0% of cocamide from natural coconut;

1.0% of glycerin from a natural vegetarian source as humectant;

0.1% fragrance of natural or synthetic origin in water; and

remainder pure water.

The named ingredients were simply mixed together and were stored at room temperature. From time to time over a period of one year, human users removed portions of the composition, rubbed the composition either directly on their bodies or between the palms of their hands to produce a foam, and cleansed various parts of their bodies. The composition was allowed to dry in place, i.e., without rinsing with a solvent of any kind. While the quantity of foam that could be produced was observed subjectively to gradually decrease during this one year test period, no discoloration or deterioration was subjectively noted.

This composition was tested to measure the average proportions of bio-based versus fossil-based content. The bio-based content was measured according to the radiocarbon dating modern concept guidelines of ASTM-D6866. The compositions were tested and the ratio reported as a percentage with the units of “percent modern carbon”. In ASTM-D6866, the material being analyzed is a mixture of present day radiocarbon (biobase) and fossil carbon (containing no radiocarbon), then the percent modern carbon value obtained correlated directly to the amount of biobase material present in the sample. The mean bio-based result for the compostion described above was 43% by weight, and the mean fossil-based result was 57% by weight.

### Alternate Ingredients

While mild, natural ingredients are preferred, some synthetic ingredients can be use in the body wash composition, preferably after such materials are pre-determined to have no adverse effects on health or wellbeing.

Antimicrobials in addition to Arnica may be used in amounts ranging from about 1.0% to about 15.0% by weight. These may include the previously mentioned oil of oregano, as well as, POLYQUATERNIUM-10, POLYQUARTERNIUM-7, and others. These should be screened for suitability before inclusion and are given by way of example but not limitation.

Alternate nonionic surfactants may be used in amounts ranging from about 0.05% to about 15.0% by weight. Such nonionic surfactants may include, by way of example but not limitation, ammonium lauryl sulfate, ammonium laurthe sulfate, cocamide propyl betaine, cocamide TEA, lauramide MEA, TERGITOL®/NP-9, TWEEN® 80 (polysorbate-20), coco betaine, and decyl glucoside,

Polyalcohols in amount ranging from about 10.0% to about 25.0% by weight may be used. These include, dipropylene glycol, tripropylene glycol, glycerin, and sunflower glycerides.

Natural oils in small amounts ranging from about 2.0% to about 7.0% by weight may be used. These include palm oil, coconut oil (cocos nuciefera), oregano oil, hydrogenated hemp seed oil, jojoba oil, neem oil, eucalyptus oil, aloe vera gel, sweet almond oil, orange essential oil, tangerine essential oil, grapeseed oil, avocado oil, and hydorgentaed jojoba oil.

Herbals as water extracts in amounts ranging from about 1.0% to about 7.0% by weight may be used for various purposes including as medicinals and fragrances. These include extracts of chamomile recutita, lemongrass oil, oregano oil, valerian, ginseng, dandelion, myrrh, spearmint, rose, calendula officinalis, sandalwood, chrysanthemum, and colloidal oatmeal. Further, pink grapefruit juice has a refreshing fragrance and may be used for its fragrance, as well as for its emulsifying effect.

The embodiments of the invention are believed to be safe to use and different from rinseless body wash products currently available in the personal care market because they are water-based and include only ingredients with no known negative health related consequences. A safe to use product is of prime importance to this invention because use may become long term in the case of war or natural disasters.

While the present invention has been described in conjunction with embodiments and variations thereof, one of ordinary skill, after reviewing the foregoing specification, will be able to effect various changes, substitutions of equivalents and other alterations without departing from the broad concepts disclosed herein. It is therefore intended that Letters Patent granted hereon be limited only by the definition contained in the appended claims and equivalents thereof.

What is claimed is:

1. A body wash composition that does not require rinsing and that topically cleanses a biological entity, comprising:
from about 0.5% to about 20.0% by weight of a concentrated extract of Arnica; and
remainder pure water.

2. The body wash composition according to claim 1, wherein the concentrated extract of Arnica is present in an amount ranging from about 2.0% to about 15.0% by weight.

3. The body wash composition according to claim 1, wherein the concentrated extract of Arnica is present in an amount ranging from about 2.0% to about 5.0% by weight.

4. The body wash composition according to claim 1, wherein the concentrated extract of Arnica is one of an infusion of Arnica flowers in pure water or a tincture of Arnica flowers in alcohol, and is present in an amount effective to provide antimicrobial properties sufficient to reduce body odor of a biological entity.

6. The body wash composition according to claim 1, further comprising from about 0.05% to about 5% by weight of a foaming agent.

7. The body wash composition according to claim 6, wherein the foaming agent is a nonionic surfactant.

8. The body wash composition according to claim 6, wherein the foaming agent is at least one of sodium laureth sulfate, cocamide DEA, cocamidopropyl betaine, cocamide betaine, coco betaine, natural coconut oil, lauramide MEA, decyl glucoside, cocamide methyl MEA, and Polysorbate 80 acetate.

9. The body wash composition according to claim 6, wherein the foaming agent is CALBLEND® CLEAR.

10. The body wash composition according to claim 1, further comprising from about 1.0% to about 15.0% by weight of a humectant.

11. The body wash composition according to claim 9, wherein the humectant includes glycerine or aloe vera gel.

12. The body wash composition according to claim 1, further comprising from about 0.25% to about 2.0% by weight of fragrance.

13. The body wash composition according to claim 1, further comprising from about 0.5% to about 10.0% by weight of at least one of Vitamin E, aloe vera gel, tea tree extract, peppermint extract, anis extract, valerian extract, lemongrass oil, calendula extract, nettle extract, lavender extract, rose extract, lemon juice, lime juice, and grapefruit juice.

14. The body wash composition according to claim 1, wherein the body wash composition has a pH value of ranging from 5.5 to 9.0.

15. The body wash composition according to claim 1, wherein the body wash composition has an appearance that Is clear and colorless, a smell that is pleasant, evaporates in 30 seconds or less, and does not clog pores of the biological entity's skin.

16. The body wash composition according to claim 4, wherein the foaming agent body produces a foam when rubbed onto hands and other body parts of the biological entity so that the foam feels wet to the biological entity and a small amount of the body wash composition covers a large area.

17. A body wash composition that does not require rinsing and that topically cleanses a biological entity, comprising, in percent by weight:
2.0% of a concentrated extract of Arnica;
1.0% of a foaming agent which is CALBLEND® CLEAR;
1.0% of cocamide from natural coconut;
1.0% of glycerin from a vegetarian source;
0.1% of a fragrance; and
remainder pure water.

18. A method of externally cleansing a body of a biological entity, comprising the steps of:
providing the body wash composition according to claim 1; and
applying the rinseless body wash composition externally to the body of the biological entity.

19. The method according to claim 18, wherein the step of applying includes applying to sensitive areas of the body including genital and anal areas even when allowed to dry in place.

20. The method according to claim 18, further comprising at least one of removing by toweling with a cloth and allowing the body wash composition to air dry.
